(19) United States
(12) Patent Application Publication
(10) Pub. No.: US 2014/0065575 A1
(43) Pub. Date: Mar. 6, 2014

(54) METHOD AND APPARATUS FOR TREATMENT OF SOLID MATERIAL INCLUDING HARD TISSUE

(71) Applicant: LASER ABRASIVE TECHNOLOGIES, LLC, Walpole, MA (US)

(72) Inventors: Gregory B. ALTSHULER, Lincoln, MA (US); Andrei V. BELIKOV, St. Petersburg (RU); Valery Y. KHRAMOV, St. Petersburg (RU)

(73) Assignee: LASER ABRASIVE TECHNOLOGIES, LLC, Walpole, MA (US)

(21) Appl. No.: 14/011,419

(22) Filed: Aug. 27, 2013

Related U.S. Application Data
(63) Continuation of application No. 12/139,994, filed on Jun. 16, 2008, now abandoned.
Application No. 12/139,994 is a continuation of application No. PCT/US2006/062190, filed on Dec. 15, 2006.
(60) Provisional application No. 60/751,109, filed on Dec. 15, 2005.
(60) Provisional application No. 60/867,281, filed on Nov. 27, 2006.

(51) Int. Cl. A61C 19/00; A61B 18/22

(52) U.S. Cl. CPC A61C 19/00; A61B 18/22
USPC 433/215

(57) ABSTRACT

An apparatus for treatment of dental tissue has a first laser source optically connected to a first channel and the same first laser source optically connected to a second channel. The second laser source is optically connected to the first channel. That second laser source is designed to be pumped via the first channel by the diode laser to generate a power of radiation sufficient to cut hard dental tissue. The second channel is connected to a device for treatment of soft dental tissue and is designed to transmit radiation sufficient for treating soft dental tissue. The first laser source can be a diode laser designed to emit radiation of a wavelength selected from a range of 700 nm to 2700 nm. The second laser source can be a solid-state or fiber laser designed to emit a wavelength from a range of 2700 nm to 3000 nm.

### RELATED APPLICATIONS

This application is a Continuation application of co-pending U.S. patent application Ser. No. 12/139,994 filed on Jun. 6, 2008, which in turn is a Continuation of PCT application serial number PCT/US2006/062190 filed on Dec. 15, 2006, which claims priority to U.S. provisional application Ser. Nos. 60/751,109 filed on Dec. 15, 2005 and 60/867,281 filed on Nov. 27, 2006, all of which are incorporated herein by reference in their entirety.

### FIELD OF THE INVENTION

The present invention relates to dental treatments, and more particularly to apparatus and method of hard and soft tissue treatment.

### BACKGROUND OF THE INVENTION

Lasers are used for advanced treatment of hard tissue and soft tissue. The main advantages of a laser for treatment of hard tissues are minimum invasiveness, painlessness, and maximum precisions of the procedure. The main advantages for treatment of soft tissues are homeostatic effect and sterilization.

Several lasers were proposed for dental hard and soft tissue treatment. Erbium (Er) lasers with wavelengths of 2690-2940 nm were proposed and used for hard tissue treatment. CO₂ lasers with wavelengths of 9300-10600 nm and excimer lasers with wavelengths of 194-350 nm can also be used for hard tissue treatment. Er lasers and CO₂ lasers can also be used for soft tissue treatment but other lasers with wavelengths of 960-2600 nm produce a better homeostasis effects. In commercial applications, only Er lasers with flashlamp pumping are used for hard tissue treatment. For soft tissue treatment, continuous wave (CW) CO₂ lasers, diode lasers with wavelengths of 800-980 nm or Nd:YAG lasers with a wavelength of 1064 nm are used. Some manufactures package an Er laser and a soft tissue laser in one box. The main disadvantages of this solution are a very high cost and a large size of the device. Another disadvantage is in using a flashlamp pumped Er laser or a CO, laser with energy delivery through an IR fiber with low transmission and limited lifetime. Alternative ways, such as delivering energy through an articulated arm or packaging a flashlamp pumped Er laser in a handpiece, are not satisfactory to a dentist, because such a delivery system is too bulky when compared to a conventional instrument or to fiber delivery. Due to this complexity, the cost of the existing dental lasers is very high and is the main limitation of a widespread use of the laser technology in dentistry.

The proposed invention provides embodiments of a laser with a quartz fiber delivery system, has overall low efficiency and can be built at a low cost. The present invention addresses the need to create a dental laser, a system and method for hard and hard and soft tissue treatment using diode laser pumping with maximum efficiency and minimum cost for better penetration of the dental market.

### SUMMARY OF THE INVENTION

The present invention is an apparatus for treatment of dental tissue comprising a first laser source optically connected to a first channel and the same first laser optically connected to a second channel. The invention also comprises a second laser source optically connected to the first channel. That second laser source is designed to be pumped via the first channel by the diode laser to generate a power of radiation sufficient to cut hard dental tissue. The second channel is connected to a device for treatment of soft dental tissue and is designed to transmit radiation from the diode laser sufficient for treating soft dental tissue. In that apparatus the first laser source can be a diode laser designed to emit radiation of a wavelength selected from a range of 700 nm to 2700 nm. The second laser source can be a solid-state or fiber laser designed to emit a wavelength from a range of 2700 nm to 3000 nm. It is also provided that the diode laser is designed to emit radiation of a wavelength selected from the range of 960 nm to 980 nm or 1350 nm to 1850 nm. Additionally, the first laser source can be a diode pumped solid-state or fiber laser and the second laser source is a solid-state laser. The second laser source can be a solid-state or fiber laser with active element doped on Erbium, Holmium, Dysprosium or Uranium ions. The diode laser can be disposed in a main unit of the apparatus, while the solid state or fiber laser can be disposed in a hand piece or outside the hand piece in the first channel. Especially beneficially in the present invention is the first channel made of a quartz fiber. To direct the radiation from the first laser source either to the first channel or to the second channel, a switch is provided.

In another implementation of the present invention an apparatus for treatment of dental tissue comprises a diode laser mounted in a main unit for generating a diode laser radiation and a first optical system for coupling the diode laser radiation to a quartz fiber. A solid-state or fiber laser is coupled to the quartz fiber and is designed to be pumped via the quartz fiber by the diode laser radiation to generate a power of radiation of the solid state laser sufficient to cut hard dental tissue. A second optical system delivers the radiation of the solid-state or fiber laser to dental tissue. The diode laser is designed to emit radiation of a wavelength selected from a range of 700 nm to 2700 nm, and the solid state or fiber laser is designed to emit a wavelength from a range of 2700 nm to 3000 nm. Also, the present invention contemplates that the diode laser is designed to emit radiation of a wavelength selected from the range of 960 nm to 980 nm or 1350 nm to 1850 nm.

The present invention also provides for an apparatus for treatment of dental tissue comprising a diode pumped solid-state or fiber laser mounted in a main unit for generating radiation. The apparatus also comprises a first optical system for coupling the radiation from the diode pumped solid-state laser to the quartz fiber, and a second solid-state laser optically connected to the quartz fiber and designed to be pumped via the quartz fiber by the radiation from the diode pumped solid-state laser to generate sufficient power of radiation of the second solid-state laser to cut hard dental tissue. The second optical system is also provided for delivering the radiation of the second solid-state laser to dental tissue.

The present invention also provides a method of generating high power pulses by a diode pumped solid-state or fiber laser. The method comprises the steps of pumping a solid-state laser with radiation from a diode laser, the pumping occurring at a power above a threshold of laser generation, and modulating either gains or losses of a resonator of the solid-state laser with a frequency corresponding to a self relaxation oscillation frequency of the solid state or fiber laser or to an obertone or to a harmonic of the self relaxation oscillation frequency of the solid-state or fiber laser, wherein a depth of modulation is lower than 50%. The depth of modulation of the gains of the resonator is +/−(5%-50%), and preferably +/−(20%-40%). The depth of modulation of the losses of the resonator is +/−(0.1%-30%), and preferably +/−(1%-10%). In the inventive method modulating the gains in is accomplished by modulating a current of the diode laser or by modulating coupling the power of the diode laser into the solid-state or fiber laser. Modulating the losses is accomplished by mounting at least one adaptive resonator minor, an acousto-optical modulator, an oscillating minor, or an electro-optical modulator in a cavity of the solid-state laser. Also, modulating the losses is accomplished by mounting a saturated transmission modulator in a cavity of the solid-state laser. The modulating frequency can be in the range from 0.1 kHz to 25 kHz. Each pulse has a duration in a range of 10 ns to 100 μs, and, preferably, from 100 ns to 25 μs.

A system for practicing the above described method comprises a diode laser, a solid state laser or a fiber laser which is pumped with radiation from the diode laser above a threshold of laser generation when the system is in operation, and a device for modulating either gains or losses of a resonator of the solid-state laser or a fiber laser with a frequency corresponding to a self relaxation oscillation frequency of the solid state or fiber laser or to an obertone or to a harmonic of the self relaxation oscillation frequency of the solid-state or fiber laser, wherein a depth of modulation is lower than 50%.

The present invention also contemplates an apparatus for treatment of dental tissue comprising a diode laser or a diode pumped solid state or fiber laser source designed to generate radiation having a wavelength from a range of 2600 nm to 3000 nm. The apparatus also comprises a focusing system disposed in a hand piece and optically coupled to the radiation. The focusing system is serving to focus the radiation into a beam spot on the dental tissue. The spot has a spot size from a size range of 3 μm to 200 μm and fluence from a range of 0.5 J/cm² to 200 J/cm². The apparatus also has a scanning system disposed in the hand piece to receive the radiation from the diode laser or the diode pumped solid state or fiber laser source to scan the spot across the dental tissue according to a treatment pattern. The treatment pattern is characterized by a fill factor area ranging from 10% to 95%, preferably from 50% to 75%. The diode pumped solid-state or fiber laser is mounted in the hand piece and a diode laser mounted in a main unit optically connected with the hand piece. Also, both the diode laser and the solid state or fiber laser can be mounted in the hand piece. The diode pumped solid-state or fiber laser can be continuous wave or quasi continuous laser with average power 0.1-70 W.

The present invention also contemplates a method for treating a material with optical radiation, the method comprising obtaining radiation from a radiation source with fluence and power density sufficient for ablating the material in a treatment zone having a first portion and a second portion. Further the method provides for applying the radiation to the treatment zone of the material to ablate the material in the first portion of the material in the treatment zone. Then the method provides for acoustically, mechanically or chemically removing the material from the second portion of the material in the treatment zone, wherein the first portion is characterized by a fill factor relative to the treatment zone is ranging from 10% to 95%. The referenced material can be dental tissue or dental material.

The method further contemplates forming an array of cavities in the first portion of the material in the treatment zone after the step of applying the radiation. The array can be periodical. The cavities range in size from 1 μm to 200 μm.

Specifically, the method contemplates that mechanically removing the material is accomplished by directing high speed particles onto the second portion of the material. The high speed particles are accelerated by the same radiation that ablates the first portion.

Also, applying the radiation to the treatment zone of the material ablates the material and results in formation of the high speed particles as products of ablation of the material in the first portion. The high speed particles are redirected to second portion of treatment zone and mechanically destroying the second portion. Applying the radiation to the treatment zone can also result in formation of an acoustic shock wave which is redirected to the second portion of the material and acoustically destroy second portions.

An optical system of for ablating a material including dental tissue comprising an input end for receiving input radiation, a body along which the input radiation propagates and transforms into a plurality of beams, and an output end for directing the plurality of the output beams onto a treatment zone to create treatment patterns on a treatment zone with a fill factor ranging from 10% to 95%. More preferably, the fill factor is 30-85%, and most preferably 50-75%. The body can comprise a plurality of optical fibers in which the input radiation propagates.

More specifically, the optical fibers are sapphire fibers. It is also contemplated that the body comprises a plurality of hollow waveguides, a plurality of focusing lenses, or a plurality of focusing mirrors. The body can comprise a scanner designed to create the plurality of microbeams by spatial scanning of one or several microbeams. The system can further comprise a reflector of products of ablation and a shock wave for redirecting the products of ablation and a shock wave to the treatment zone.

The present invention also provides for an opto-mechanical system for processing a material including dental tissue, the opto-mechanical system comprising an input end for receiving input radiation, a focusing system for focusing the input radiation into a spot having a spot size, a channel for delivering abrasive particles to the spot, each particle having a size smaller than the spot size, and an opening for directing the particles accelerated by the input radiation toward a treatment zone on the material.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1a and FIG. 1b are a schematic illustration of one of the embodiments of a dental laser system.

FIG. 2 is a schematic illustration of one of the embodiments of a dental laser system.

FIG. 3 is a schematic illustration of one of the embodiments of a dental laser system.

FIG. 4 is a graph showing of a temporal profile of the laser emission power with a periodical sequence of pulses with a period T=2π/Ω and pulsewidth τ<T generated by a solid-state or fiber laser with quasiresonance modulation of laser resonator losses.

FIG. 5 is a schematic drawing of one possible optical device for cavity losses modulation using an output oscillation mirror.

FIG. 6 is a graph showing computer-calculated temporal output power dependences of Er:YLF laser pumped by 500 W laser diode radiation.

FIGS. 7a and 7b are a schematic illustration showing a possible embodiment of a laser handpiece with a CW or QCW laser system and a scanner for dental tissue treatment.

FIG. 8 is a graph showing a pattern of cavities on a dental tissue after laser treatment.

FIG. 9 is a schematic illustration of one of the embodiments of a laser dental system for treatment of soft and hard tissue.

FIG. 10 is schematic illustration of a cross section of one of the embodiments of a handpiece for a dental laser system through the center of the fiber unit.

FIG. 11 is a schematic illustration of a cross section one of the embodiments of a handpiece for a dental laser system through the center of the tube.

FIG. 12 is schematic drawings of the diode laser with coupling output energy into fibers.

FIG. 13 is cross-section view of optical schematic of the diode pumped solid-state laser.

FIG. 14 is a schematic drawing of the diode pumped solid-state laser.

FIG. 15 is optical tracing of pumping radiation in a diode pumped solid-state laser.

FIG. 16 is a schematic illustration of one of the embodiments of a laser abrasive tip.

FIG. 17 is a schematic illustration of treatment patterns on treated tissue.

FIG. 18 is a schematic illustration of one of the embodiments of a tip with a particle recycler.

FIG. 19 is a schematic drawing of the particle recycler and an optical system for creation of plurality of focusing beams on a treated material or hard tissue.

FIG. 20 is a schematic illustration of a tip design with particle and shock wave reflectors.

FIG. 21 is a schematic illustration of one of the embodiments of a tip.

### DETAILED DESCRIPTION OF THE PREFERRED EMBODIMENTS

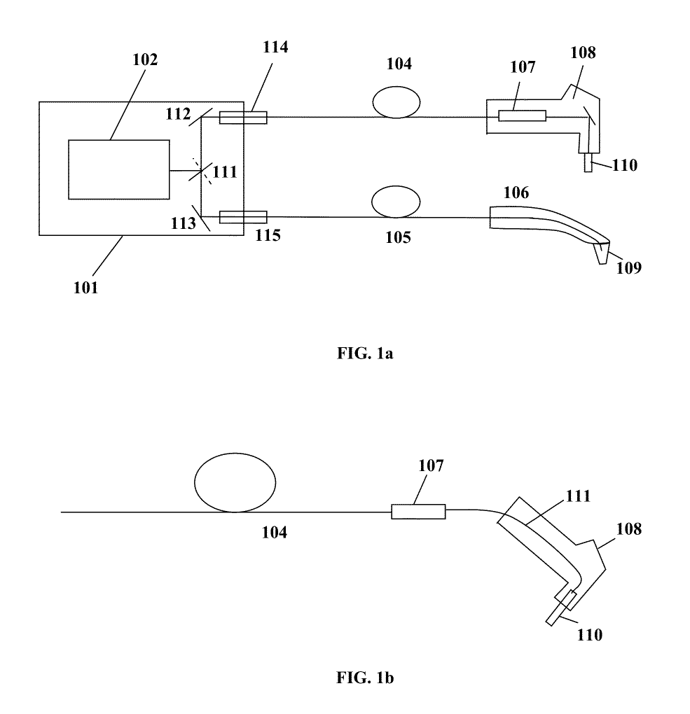

A dental laser system shown in FIG. 1a and FIG. 1b are a possible embodiment and is not intended to be limiting in any way. A main unit 101 comprises of the first laser, which is a diode laser 102, a power supply, control electronics, a cooling system, and other components necessary for functionality of the laser and hand pieces. Diode laser wavelengths of 700-2700 nm are suitable for transmission through a standard quartz fiber having a length of 0.25-2 m without significant energy losses. Laser energy from the main unit 101 is delivered through an umbilical 105 or 104, comprised of a quartz fiber, to a soft tissue hand piece 106 or a hard tissue hand piece 108. The hard tissue hand piece 108 is comprised of a light frequency converter 107, a treatment tip 110, and optical, mechanical and, optionally, electrical components. The light frequency converter 107 converts the wavelength (frequency) of the diode laser to wavelengths which are most effective for hard tissue processing and/or for accelerating abrasive particles for hard tissue processing. The converter can be a coherently pumped laser or a non-leaner optical converter. The range of wavelengths for hard tissue processing is 2650-3000 nm or 9300-10600 nm. In one embodiment, the fiber 104 can be a fiber laser and function as a light frequency converter. The hand piece 108 comprises of the tip 110, and optical, mechanical, electrical, water and air delivery components. The optical components deliver light to the soft tissue through the tip 109 without converting the wavelength. Radiation emitted from the diode laser 102 can be used for soft tissue treatment and for pumping the frequency converter 107. Switching between the modes of operation can be performed by an optical switch 111. Diode laser radiation can be directed through coupling elements 112 and 113 into the quartz fibers for delivery of the radiation to the hand pieces. Alternatively, two different hand pieces may be connected with the main unit through connectors 114 or 115. The frequency converter 107 can be mounted in a part of the umbilical close to the hand piece as shown in FIG. 1b. Radiation from the converter 107 is delivered to the tip 110 through a short fiber 111 which can be a quartz, hollow, sapphire, fluoride, or germinate glass fiber.

Experiments have shown that ablation of hard and soft tissues is achieved best when the fluence of laser pulse radiation is in the range of 1-200 J/cm² and pulse durations are in the range of 0.5-1000 μs. A typical spot size for dental hard tissue treatments is 0.2-1 mm. Thus, the energy per pulse must be in the range of 0.1-1500 mJ. For practical reasons, the speed of hard tissue removal must be in the range of 0.5-1 mm³/s. Accordingly, a laser has to run in a repetition rate mode with a repetition rate of 5-100 Hz. The teachings of a laser abrasive method of processing hard tissue, which requires 2-6 times lower energy for ablation than the direct laser method, are disclosed in U.S. Pat. Nos. 7,048,731, 6,558,372 and 6,709,269, which are incorporated herein by reference.

An analysis of the laser abrasive processes shows that the average efficiency of tissue removal may be preserved if instead of the quasi-CW radiation, which it is typical for solid-state lasers operating in a free-running mode, a regular sequence of laser pulses is used. Each pulse in such a sequence has to remove some definite volume of the hard tissue material. The fluence of each pulse will exceed 1 J/cm² when energy pulse is greater than 0.01-10 mJ for the beam spot size in the range of 30-100 μm and the pulse width in the range of 0.01-10 μs. The laser abrasive method may also be used to increase the efficiency of ablation. For a typical size of the beam on the tissue of about 250 μm, the minimum peak power is 500 W and energy per pulse is 0.5 mJ with the pulse width of about 1 μs.

Below are descriptions of possible embodiments for practicing of the present invention. The embodiments are provided as illustrations only and are not intended to be limiting in any way.

In one embodiment, the radiation of the diode laser 102 is coupled into the fiber using micro-optics or another optical system, such as a duct lens. The diode laser has a wavelength of 700-2700 nm. Such a wavelength is suitable for pumping of the wavelength converter and is transmittable through a quartz fiber. The wavelengths converter 107 is a solid-state laser with emitting wavelength in the range of 2700-3000 nm.

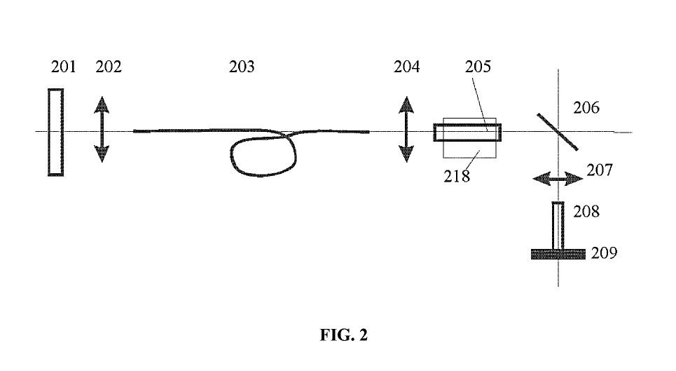

The diode laser represents a laser bar or a stack of laser bars, lasing radiation with a wavelength suitable for further pumping of the solid-state laser. A possible optical schematic of this embodiment is shown on FIG. 2 and is not intended to be limiting in any way.

Pumping radiation from a laser bar or bars 201 passes through a focusing lens 202 and coupling by an optical waveguide 203. An optical system 204 adjusts the mode of distribution of output waveguide radiation with a hand piece laser 205 mode distribution to obtain high efficiency of conversion of pumping radiation. The mirrors of the laser 205 are covered on active rod faces directly. The output radiation of laser 205 with a wavelength in the range of 2700-3000 nm passes straight or through an optical folded system 206, a focusing lens 207 and an optical waveguide 208 and illuminates a target surface 209. The active element of the laser 205 comprises of a cooling system 218. Cooling of the laser 205 can be provided by water or air flow from the main unit.

The pumping radiation source 201 may be a diode laser with the output wavelength of about longer than 700 nm. To pump Er³⁺-ione doped laser active media (Er:YAG, Er:YSGG, Er:YLF, Er:YAP:Er, EnBaY₂F₈, etc.) the wavelength of the laser diodes may be in the range of 960-980 nm or about 1500-1600 nm. To pump Ho³⁺-ione doped laser active media (Ho:YLF, Ho:BaY₂F₈, LiHo₁₋ₓPrₓF₄, NaHo₁₋ₓPrₓF₄, etc.) the wavelength of the laser diodes may be in the range of about 800-900 nm or about 1100-1150 nm. The optical system 202 may be a micro-optical system for coupling of light from a laser bar to the fiber (such as the one produced by LIMO GmbH), optical lenses or duct lenses. The active elements 205 may be any suitable crystal activated by ions of Er, Ho, Dy, U with the necessary 2700-3000 nm range laser transitions (for example, Er:YAG, EnYSGG, Er:YLF, Er:YAP:Er, Er:BaY₂F₈, Ho:YLF, Ho:BaY₂F₈, LiHo_{1-X}PrₓF₄, NaHo_{1-X}PrₓF₄, etc.). The folded unit 206 may be a flat, spherical, aspherical or metallic mirror. The focusing system 207 may be combined with the folded system 206. The optical waveguide 208 can be a quartz, sapphire, ceramic or hollow fiber. The optical waveguide 203 is a quartz waveguide and may comprise of some separate waveguides having diameters of 0.1-1 mm. Pumping radiation may be coupled by each waveguide of separate laser diodes or a set of laser diodes. End of pumping of the solid-state laser, side pumping or a combination of both pumping methods can be used individually or simultaneously.

Resonator minors of the solid-state laser may be fabricated on faces of the active rods 205 or installed near the facet of the active rod. At least one such minor can be used for modulation of the resonator loss. Such modulation can be provided by an additional modulator placed between the mirror and faces of the laser element.

In the above-described embodiment, the diode laser light may also be used for soft tissue treatment.

In another embodiment, the diode laser 102 is a diode pumped solid-state laser having wavelengths in the range of 700-2700 nm. Such wavelengths are suitable for pumping of the wavelength converter and are transmittable through a quartz fiber. The wavelengths converter 107 is a solid-state laser with emitting wavelength in the range of 2700-3000 nm.

In this embodiment, laser diodes pump the intermediate laser converter placed as laser diodes in the main housing. The intermediate laser converter is a solid-state laser which possesses a low divergence angle of output radiation. For this reason, expensive and complex micro optics are not necessary to guide the intermediate laser converter's radiation by an optical fiber. The wavelength of the intermediate laser has to be suitable for pumping of a second laser which is placed in the hand piece and converts the radiation of the intermediate laser in the needed range of 2700-3000 nm. For example, an Er:glass laser having a wavelength of 1540 nm pumped by laser diodes having a wavelength of 950-970 nm can be used as an intermediate converter for pumping of the laser placed in the hand piece and based on Er-doped crystals (Er:YAG, Er:YLF, etc.). In another example, an Nd:YAG laser having a wavelength of 1120 nm and pumped by laser diodes having a wavelength of 810 nm can be used as an intermediate converter for pumping of a laser based on Ho-doped crystals.

The above-described embodiment can have a great advantage, if stocks losses in the hand piece laser converter of intermediate radiation are small. In order to obtain radiation with a wavelength of about 3000 nm, an active media based on U:LiYF₄(transition ⁴I_{11/2}-⁴I_{9/2} of U-ions) or Dy:BaY₂F₈ (transition ⁶H_{15/2}-⁶H_{13/2} of Dy-ions) crystals can be used. The Tm:YAG laser generating a wavelength in the range of 1950-2000 nm (transition ³H₄-³H₆ of Tm-ions) or about 2300 nm (transition ³F₄-³H₅ of Tm-ions) can be used as an intermediate converter for pumping of the U:LiYF₄ or Dy:BaY₂F₈ crystals. It is significant that the 3000-nm laser operates as a quasi four-level system because the pumping radiation excites the high Stark sublevels of the upper laser level and the generation occurs between low Stark sublevels of the upper laser level and high Stark sublevels of the low laser level. The low laser level is the ground level for the laser media of both hand pieces; however, the energy gap is greater than 1000 cm⁻¹ between the low and the high Stark sublevels of a ground level.

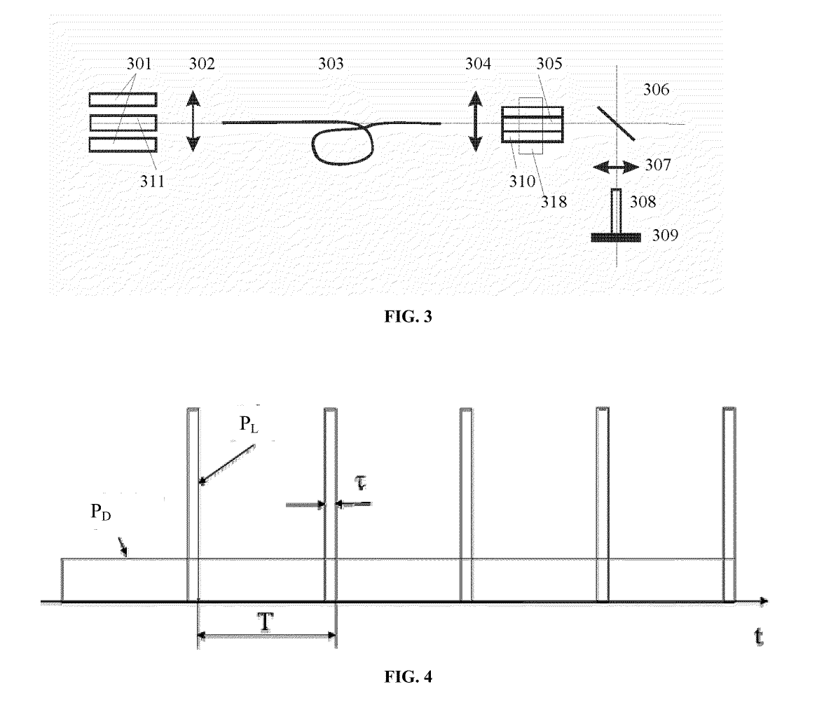

An optical schematic shown in FIG. 3 is one possible embodiment and is not intended to be limiting in any way. Radiation of diode lasers 301 pumps an intermediate solid-state laser converter 311 which has minors covered on its facets or with at least one minor separated from the laser element. At least one such minor can be used for modulation of the resonator loss. Such modulation can be provided by an additional modulator placed between the mirror and the facet of the laser element. Several diode bars or diode lasers can be used for pumping. Laser light from the intermediate laser converter passes through a focusing lens 302, an optical fiber 303 and an optical lens 304. The latter is placed in the hand piece and shapes the laser beam for pumping of the hand piece laser converter medium. Additional optical elements 310 are positioned around laser medium 305 to better adjust pumping radiation distribution with the laser converter modes. The minors of the hand piece laser converter are covered on the laser element facets. Laser radiation with a wavelength in the range of 2700-3000 nm passes straight or through an optical folded system 306, a focusing lens 307 and an optical waveguide 308 and illuminates a target surface 309. The active element 305 is connected with a cooling system 318. The laser light from the first solid-state laser 311 can also be used for soft tissue treatment.

In yet another embodiment, the diode laser 102 is a pumped fiber laser with wavelengths in the range of 800-2700 nm. Such wavelengths are suitable for pumping of the wavelength converter and are transmittable through a quartz fiber. The light frequency converter 107 is a solid-state or nonlinear optical wavelength converter, i.e. an optical parametric oscillator. In contrast to the above-described embodiments, in this embodiment a fiber laser pumped by laser diodes can be used as an intermediate converter. The fiber laser may be comprised of one or more guides to increase the output radiation power and to obtain more uniform pumping of the hand piece laser converter. The output fiber laser's wavelength has to be adjusted to the optimal wavelength used for pumping of the hand piece converter and may be, for example, 1120 nm for a Ho-doped hand piece converter medium or 1500-1600 nm for an Er-doped medium. It can also be a Tm doped fiber. The fiber laser may also include a fiber Raman shift converter. The fiber laser can operate at a short duration pulse mode. In this case, the hand piece laser converter can be set up as an optical parametric oscillator to produce output radiation in the range of 2700-3000 nm or 9600-10600 nm. The fiber laser can be made of an Er-doped material which is transparent for 2700-3000 nm, i.e. fluoride or germanium glass. In this case, the wavelength converter 107 is combined with the fiber delivery system 105 into one component.

In another embodiment, a method of resonance modulation of gain or loss of a diode-pumped solid-state or a fiber laser to increase output power is disclosed. To increase output peak power of the hand piece solid-state laser converters described above, a quasi resonance modulation mode of the laser can be used. Resonator losses or resonator gains can be used for the quasi resonance modulation of the laser parameters.

Such modulation with a frequency Ω will provide modulation of output laser emissions with the same frequency Ω, their harmonics or obertones. A temporal profile of the laser emission is shown in FIG. 4 and can be a periodical sequence of pulses with a period T=2π/Ω and a pulse width τ<T.

Laser power P_{L} from a wavelength converter can be calculated as P_{L}=P_{D}·η·T/τ, where η is the efficiency of conversion of diode laser energy to energy of the wavelength converter (for example, an Er laser). Without modulation, T=τ and P_{L}=P_{D}·η. The maximum value of η is quant efficiency of wavelength conversion. For example, if the diode laser wavelength is 970 nm and the Er laser wavelength is 2940 nm, then the maximum value of η=0.97/2.94=0.33. In this case, P_{L} is less than 0.33P_{D}. If the laser power required for hard tissue treatment is about 500 W, then the diode power has to be greater than 1500 W and requires 10 diode laser bars with power per bar of about 150 W. A significant number of laser bars increases the cost of the system due to the cost of diode lasers and complexity and cost of fiber coupling optics. Modulation of losses of the solid-state laser can decrease the required number of bars in T/τ times if T is significantly less than the lifetime of inversion of the solid-state laser. Usually, such modulation must be very deep and be close to 100%. A modulator with 100% modulation of losses is complex, expensive and usually requires high voltage for control which can be a significant limitation for a modulator in a dental handpiece due to electrical safety and over size. To resolve these problems, the present invention proposes to use a modulator with a frequency Ω close to resonance frequencies of the solid-state laser Ψ_{N}. Such a mode of operation is defined as quasiresonance operation mode. These frequencies can be calculated using the following formula:

$${Ψ}_{N} = N \cdot Ψ = N \cdot \frac{1}{2 π} \sqrt{\frac{1}{{T}_{1} \cdot {τ}_{c}} \cdot ( \frac{{W}_{P}}{{W}_{TH}} - 1 )}$$

where N=( . . . 2, 1, ½, ⅓ . . . ) is an arbitrary parameter which can be either a whole number greater than zero or its reciprocal, Ψ is the frequency of self relaxation oscillation of the solid state laser, T₁ is longitudinal relaxation time of the active media, τ_{c} is the average lifetime of photons in the resonator, W_{P} is the pumping rate, and W_{TH} is the laser threshold pumping rate. The parameter N determines the ranges of frequency modulation for which the laser generation pulses possess the regular sequence with a very high peak power. The depth of modulation amplitude losses is about +/−(0.1%-30%), and preferably +/−(1%-10%). This depth of modulation can be achieved with low-cost modulators.

The ranges of Ψ values depend on active media and laser cavity parameters as well as on the relation between the pumping power threshold and the pumping power. An analysis of the formula for Ψ_{N} shows that the main parameters which determine the laser Ψ value are T₁ longitudinal relaxation time of the active media, and N, which determines obertone values. For lasers based on Er and Ho-doped active media, the range of self relaxation oscillation frequency is between about 25 kHz (Er:YLF, T₁=4*10⁻³ s) and 120 kHz (Er:YAG, T₁=10⁻⁴ s). To get the high peak power of pulses it is necessary for the pumping pulse duration to be as long as possible. However, for high-energy efficiency, it is also necessary that the pumping pulse duration be less than T₁. Thus, one can skilled in the art can determine the best range to be about 10-25 kHz for Er:YAG and about 0.25-25 kHz for Er:YLF. The upper limit of these ranges is determined approximately by the low threshold limit value of necessary laser radiation peak power pulse.

Implementing the laser resonator loss modulation method is possible by installing into the laser cavity an optical unit that inserts small periodical losses for laser cavity. Below are several of many possible kinds of such an optical device:

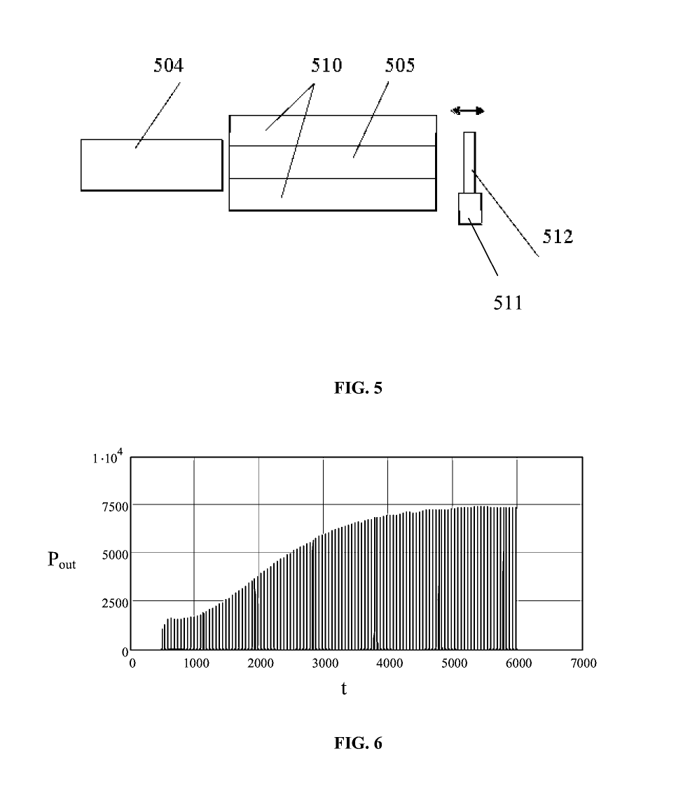

1) A schematic drawing of on possible optical device is shown in FIG. 5. The device comprised of an output coupler minor 512, a handpiece laser element 505 with controlled tilted angle of the coupler which is made as a flat semitransparent minor, a coupling optical system 504 and an added optical system 510 situated around a laser element 505 to improve pumping radiation absorption in the laser element 505 or the radius of minor with controlled curvature (a so-named adaptive mirror). The mirror 512 is mounted on a vibrator 511 which provides movement of the mirror with frequency Ω, misalignment of laser resonators and modulation of their losses. The vibrator may be an electromagnetic or piezoelectric device.

2) An acousto-optical shutter suitable for operating in the 3000 nm wavelength range.

3) An optical element such as element in point 512 but installed as a folded mirror in the laser cavity.

4) An electro-optical modulator based on suitable Pockels cell to control intracavity beam polarization.

5) A saturated absorbed shutter for the 3000 nm wavelength range (for example, based on water vapor or semi-conductor materials).

6) An optical modulator based on the effect of total internal reflection.

In order to realize modulation of a laser resonator gain it is possible to control injected current to pump the laser diodes which, in turn, are used for pumping of the solid-state laser or fiber laser. In this case, the optimum amplitude of the current modulation has to be in the special range of +/−(5%-50%), preferably ±(20%-40%) of the average current value only. CW laser diodes can be used if the threshold value of the injected current is not exceeded. In other embodiments, modulation of a solid-state laser resonator gain can be controlled by modulation of coupling energy from the pumped laser into the solid-state active media.

FIG. 6 shows a temporal profile of output power Pout (in Watts) for an Er:YLF laser as a function of time t (in μsec). This laser is pumped by a laser diode with the total power of 500 W on the wavelength of about 970 nm. The frequency of modulation is Ω=20 kHz, the depth of modulation is 2%. As one can see from FIG. 6, such a small modulation of losses leads to 100% modulation of the laser power. The full energy of pulse generation is Eₛ=290 mJ. The number of pulses in the periodic sequence is N=111. The length of the first pulse in the sequence is 1.0 μs, and the length of the last pulse is 0.35 μs. Such a temporal profile is optimum for hard tissue ablation because the pulse length in every micro pulse is shorter than the thermo relaxation time of the layer of hard tissue where the laser energy is absorbed. This temporal profile gives additional increase in ablation efficiency compared to a randomly modulated temporal profile of the same laser in free running mode.

The minimal size and price of the dental system can be achieved by using continues wave (CW) or quasi-continuous (CW) wave laser system. Such a laser can be a diode laser with a laser bar or one emitter, a diode pumped solid-state laser as describe above, or a fiber laser. Because the power of such a CW system is low, the laser beam must be focused on the treatment tissue or, in the case of the laser abrasive method, accelerated particles must be focused in a very small spot comparable with the size of an abrasive particle. The minimum power on the tissue can be calculated based on the following formula:

$$P \approx \frac{F \cdot π \cdot {d}^{2}}{4 \cdot TRT}$$

where F is the minimum fluence for ablation, TRT is thermal relaxation time of a tissue layer having thicknesses equal to the light penetration depth. The minimum fluence of ablation of a dental tissue for a microsecond range of pulsewidth is about 1 J/cm². The maximum fluence which provides saturation of efficiency of tissue ablation is around 50-200 J/cm². TRT can be calculated using the following formula:

$$TRT \approx \frac{{h}^{2}}{4 \cdot α}$$

where h is the depth of penetration of laser light into the treatment tissue. For an Er laser with having wavelength in the range of 2650-3000 nm, the depth is h≈5-15 μm. α is the thermal diffusivity

$${α}_{enaml} \approx 0.004 \frac{{cm}^{2}}{sec} .$$

For the minimum spots size d≈3-50 μm the power of an Er laser can be in the range of P≈0.1-70 W. Such power can be generated with a laser system, such as a diode laser with a bar or one emitter, a diode pumped solid-state laser as describe above, or a fiber laser. For example, it can be the system shown on FIG. 2 where the pumping laser is a 5-30 W diode laser (one emitter) with the wavelength of about 970 nm or about 1500 nm. Irradiation of this laser can be coupled into the fiber easier than irradiation from a laser bar. A solid-state laser pumped by this diode laser can be mounted in the handpiece. Alternatively, the solid-state laser can be pumped by a diode laser or a laser bar placed in the handpiece.

For effective ablation of the tissue, a small laser beam must scan across the treatment tissue with a high speed which provides effective treatment time of the area comparable with a spot size shorter than the TRT. The speed of the scanning of the beam is v>d/TRT. In our case, this speed is in the range of 5-100 cm/sec. The handpiece must be equipped with a micro scanner to provide scanning of the beam across the treatment tissue.

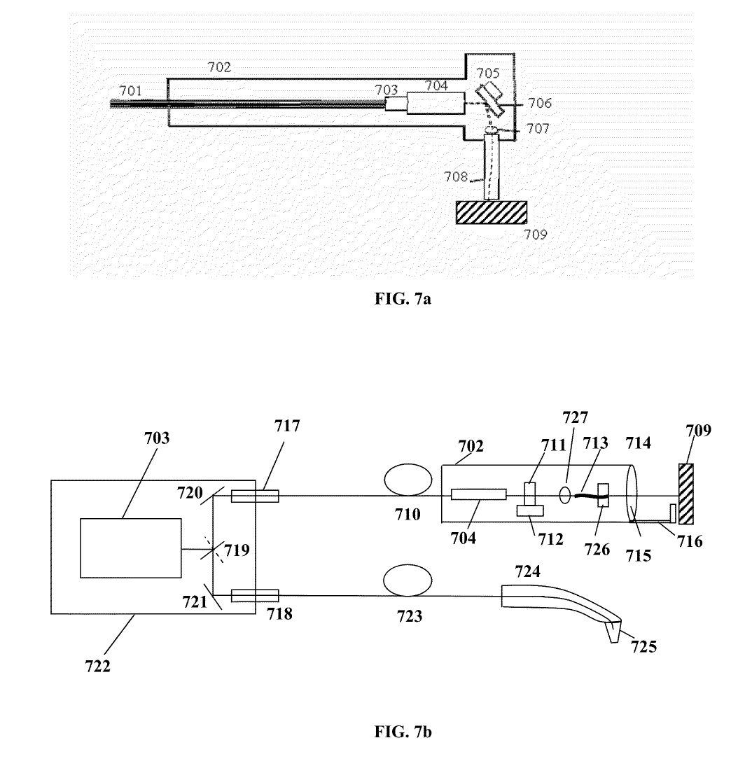

FIG. 7a shows an embodiment of a laser handpiece which comprises of a delivery system 701 which further comprises of electrical wires, and other components necessary for delivery into the handpiece. There is a diode laser 703, which can be a diode laser or a laser bar pumping a solid-state laser 704. The output beam is reflected from a mirror 706 which is connected to a motor or a piezo-element for beam scanning. The beam is focused on the tissue and is delivered to a treated tissue 709 via an optical system 707 and via a tip 708 or a free space. The types of the diode laser and solid-state lasers are described above. FIG. 7b shows an embodiment of a low-power CW or QCW laser system for dental tissue treatment. A main box 722 comprises of a diode laser 703, an optical switcher 719 to direct the diode laser radiation through coupling elements 720 and 721 into a quartz fiber 710 or 723 which further delivers the diode laser radiation to a hard tissue handpiece 702 or a soft tissue handpiece 724. The hard tissue handpiece 702 comprises of a wavelength converter, for example, an Er:YLF laser with an output minor 711 mounted on an electromagnetic or piezoelectric oscillator 712, a focusing system 727, an optical fiber 713 with the output end mounted on a scanner 726, an image optical system 715, a spacer 716, which touches the treated tissue 709. The soft tissue handpiece 724 comprises of a tip 725.

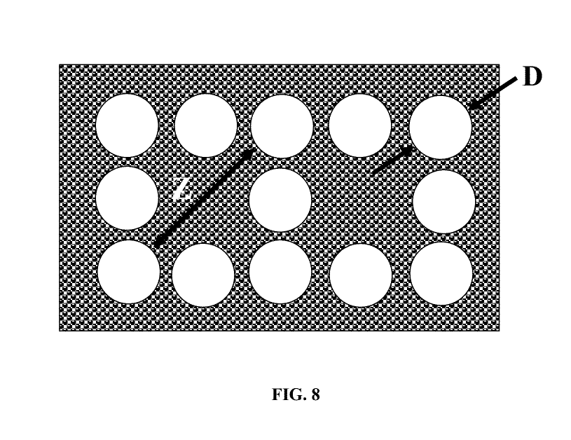

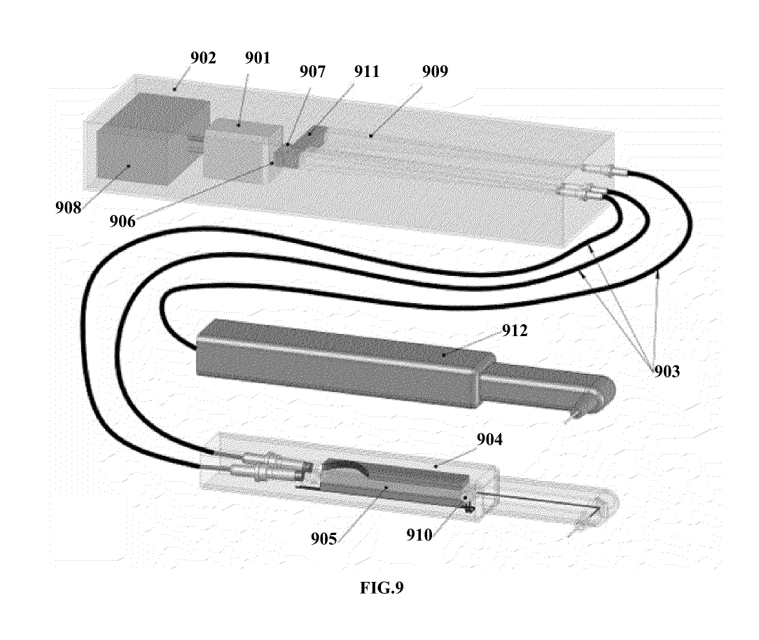

Scanning coverage or scanning pattern of the hard tissue does not need to be continuous. As shown in FIG. 8, if the distance between treatment spots Z is smaller than 100 μm then the residual hard tissue between the drilled holes with a diameter D can be easily destroyed mechanically or chemically. For such a method of scanning, the total speed of drilling will be increased due to the lower volume of tissue that needs to be ablated. The ratio of the total drilled area to the total treatment area is a fill factor. The fill factor has to be in the range of 10-95%, preferably 30-85%, and most preferably 50-75%.

One of several designs of the above system is shown in FIG. 9. Er:YLF may be used as active medium in the laser located in the handpiece. As shown in FIG. 6, in order to obtain the output energy of 200-300 mJ it is necessary to use pumping laser diodes with output power in the range of 450-700 W and total pulse duration of a sequence of micro-pulses in the range of 5-7 ms. The above-described quasiresonance modulation mode can be used to increase peak power of the main laser converter. The burst of pulses must contain 60-200 pulses to comply with the above-mentioned requirements for peak power and energy. The modulation frequency may be in the range of 8-25 kHz. The modulation frequencies in this range can be used to decrease the depth of the loss of the modulation amplitude down to only 2-5%. The average output power of the laser converter may be within the range of 3-9 W if the repetition rate of the pulse is be in the range of 1-100 Hz.

FIG. 9 shows a schematic drawing of one of several embodiments of a dental system and is not intended to be limiting in any way. The dental system comprises of a pumping laser 901, located in a main unit 902, one or more fiber units 903, and a hand piece 904 for hard tissue treatment. The pumping radiation is transferred via the fiber(s) unit 903 to the hand piece 904 for soft tissue treatment. The fiber unit 903 may be implemented as a quartz fiber or as a part of the corresponding fiber laser. The radiation emitted by the pumping laser 901 may travel through an optical system 911 and two duct lenses and fibers 903 to the hand piece 904, comprising of an additional converter 905. The converter 905 transforms the radiation emitted by the pumping laser 901 with a wavelength of about 970 nm to the radiation with a wavelength of 2810 nm. Cooling of the pumping laser 901, of the converters 905 and of the tooth surface may be accomplished with water, air, and evaporative cooling from Freon. The pumping laser 901 comprises of at least a diode laser 906 and, optionally, a converter 907.

FIG. 12 shows a detailed schematic drawing of the of diode laser 901 and is not intended to be limiting in any way. A stack of 4-10 diode laser bars having a length of 1 cm and a pitch of 0.5-1.5 mm is mounted on a heat exchanger 1207 cooled by water or overcooled gas flow 1202. Electrodes 1201 supply electrical power to the laser diodes. The radiation from a diode laser bar is coupled into two duct lenses with the input size that covers the output size of the stack of laser bars. Duct lenses 1205 can concentrate the diode laser radiation into a spot with a size of about 0.8-1 mm and numerical aperture confined to the numerical aperture of a quartz fiber with polymer cladding. Input ends of the fibers are mounted in connectors 1204.

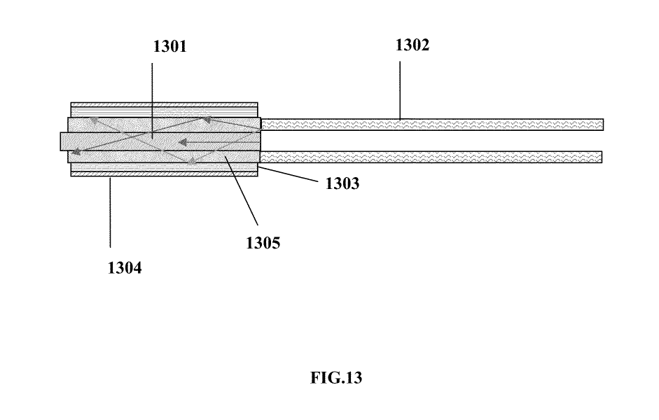

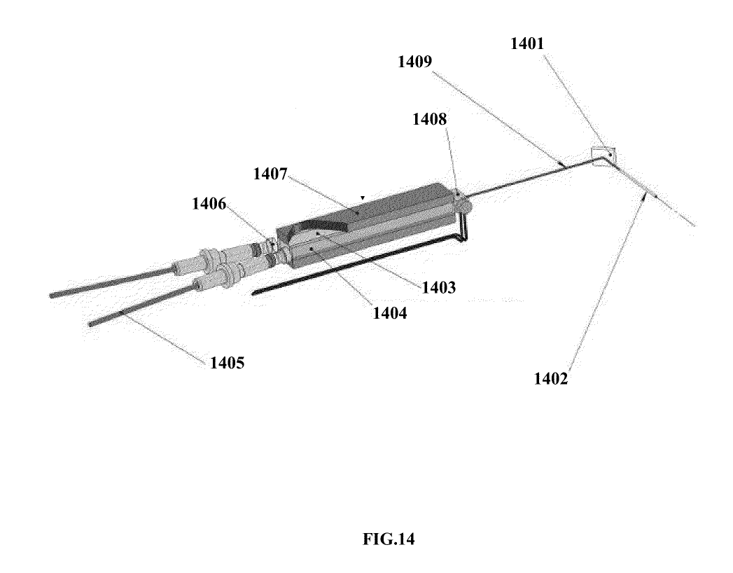

FIG. 13 shows a cross section of one possible embodiment of the solid-state laser 905 and is not intended to be limiting in any way. The laser comprises of an active element 1301 as a slab or a rod with minors located on the facets. Dielectric plates or a hollow cylinder 1305 is placed around the rod with optical and thermal contact with the active element. This assembly is housed in a tube 1304. The gap between 1305 and 1304 is used for cooling of the laser by liquid flow 1303. FIG. 14 shows the same laser 901 with a resonator modulator 1408. A slab active element 1404 is housed between two dielectric plates 1403 with the refractive index lower than the refractive index of the active element 1404. This module is housed in a cooling element 1407. Pumping radiation is delivered through a quartz fiber 1405 and an optical system 1406. Output radiation is delivered to a tip 1402 through a minor 1401. FIG. 15 shows an optical tracing of this module including an Er:YLF active element 1504, sapphire plates 1503, an optical system 1502 and a fiber 1501. One can see that the pumping radiation propagates into the module in a manner similar to the fiber laser and provides a very uniform distribution inside the active element. The main unit 902 may further comprise of a power supply, a cooling system, and control electronics in a module 908.

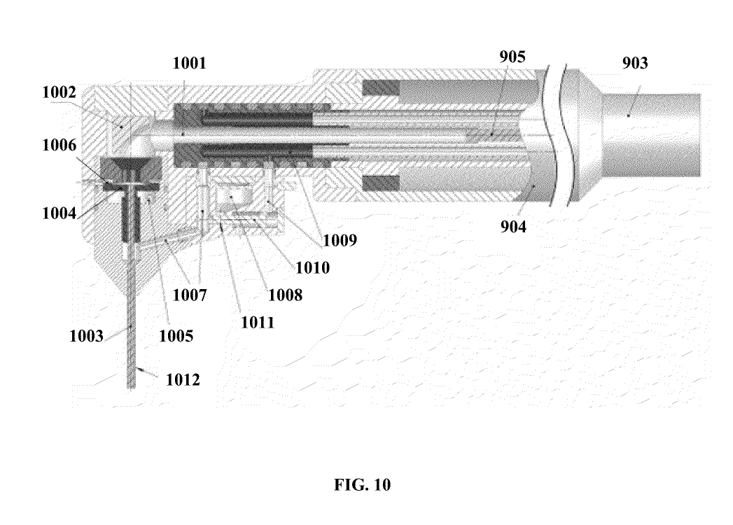

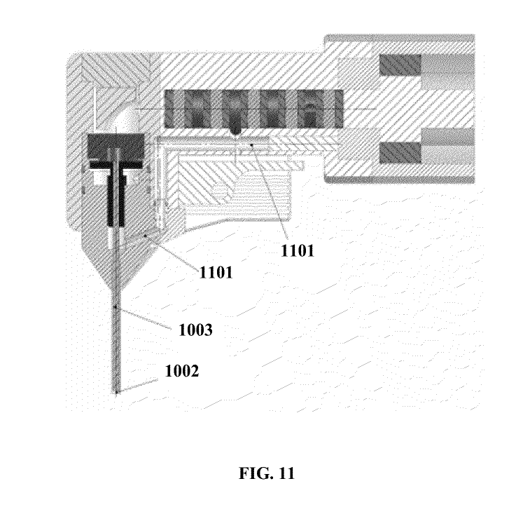

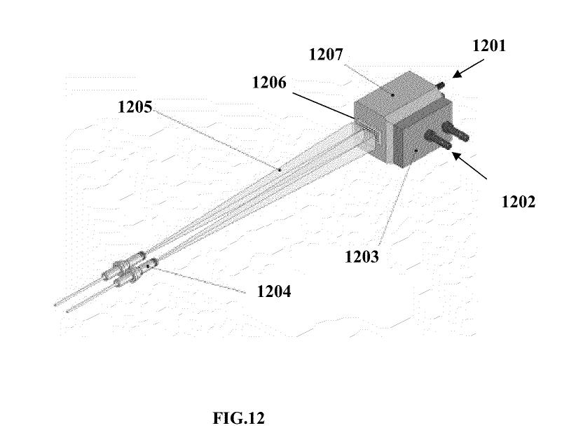

Detailed schematics of the hand piece 904 are shown in FIGS. 10 and 11 and are not intended to be limiting in any way. The hand piece functions as follows. A laser pulse, traveling from converter 905 through a delivery system 1001 hits the surface of an optical element 1002 and then the entrance of a tip 1003. Delivery system 1001 can be realized as a fiber or a hollow tube. The optical element 1002 may be a minor (or an assembly of many minors, such as a raster) and is used to control the rotational direction of a laser beam and to focus it on the tip 1003. Upon exiting the tip 1003, the laser pulse hits the surface of a treatment material, for example, a biological tissue, including, but not limited to enamel, dentin, and/or bone. During the influence of the laser pulse, the tip 1003 is in contact with the biological tissue. Immediately after the laser pulse, air, delivered via a tube 1005, is applied to a mount 1004. The tip 1003 begins to move away from the biological tissue within the mount 1004 under the air pressure. The air feed may be stopped (i.e. the air pressure in the tube 1005 is equal to atmospheric pressure) when the tip 1003 travels a predetermined distance (approximately 100-500 μm, and most preferably 200 μm), as shown in FIG. 11, away from the surface of the biological tissue. The tip 1003 then returns to its original position under the action of a spring 1006. During the time interval between the ascent and descent of the tip 1003, water is fed via a tube 1101 into a cavity 1102, created between the surface of the biological tissue and the outside end of the tip 1003 (FIG. 11). The length of the water pulse is controlled by opening/closing of the corresponding valve (not shown). The water pulse is followed by an air pulse, traveling via a tube 1007. The length of the air pulse is controlled by opening/closing of the corresponding valve (not shown). Abrasive particles (for example sapphire particles having a size of 1-100 μm) are stored in a reservoir 1008 and may be delivered during the action of the air pulse into the channel 1007. Under the action of air moving along a tube 1009, a piston 1010 with a particle container 1011 begins to move. The container 1011 with particles is situated in the air current created in the tube 1007. The air current captures the particles and empties the container 1011. Water, air and the particles reach the surface of the treatment material via a gap created by the tip 1003 and a metallic tube 1012. At the time when the laser pulse begins, the water, air and particle pulses may end, and the tip 1003 may be in contact with the biological tissue. The assembly including 1003-1008, 1010, 1011, 1101 may be disposable. Alternatively, only a part of this assembly, such as the tip 1003 and the reservoir 1008 with abrasive particles, may be disposable.

Decreasing the laser power in order to deliver the output energy sufficient for tissue ablation is the most effective way of building a low-cost dental system. Improvements of the method of ablation and tip design may increase efficiency of ablation and decrease the necessary laser power. In the present invention a new tip design and a method of ablation are described. The new method and design can be combined with the laser systems described above, but are not limited to these systems.

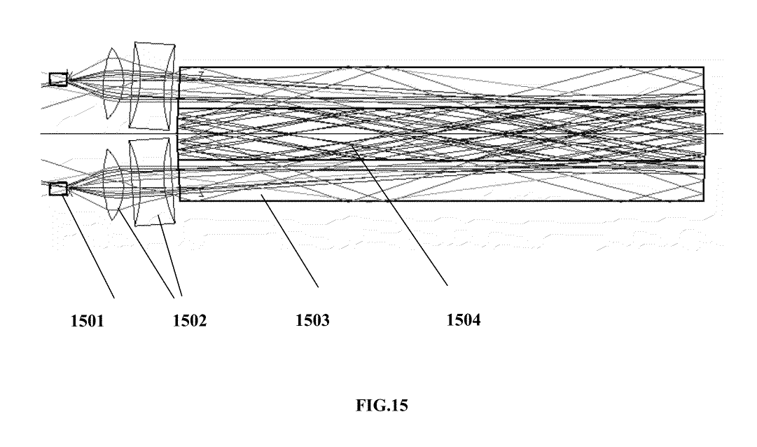

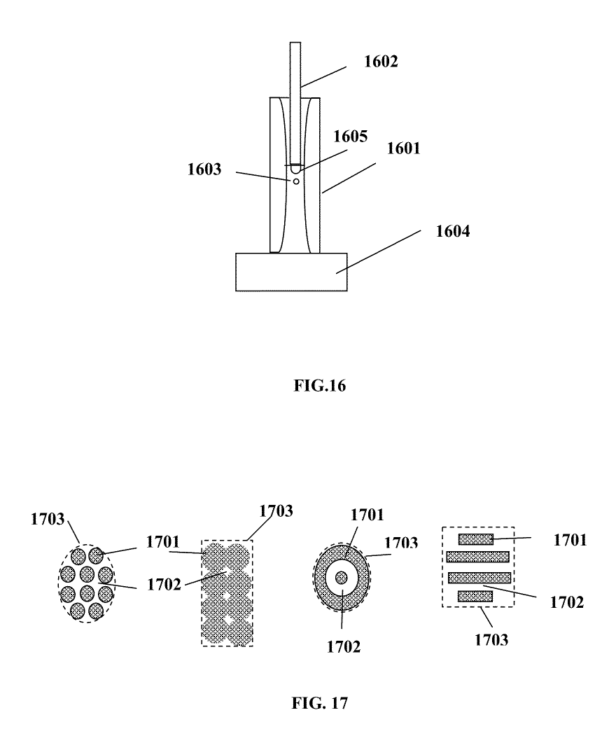

The above-described low-power CW and QCW lasers with peak power of about 3-70 W can be used for accelerating abrasive particles with the laser abrasive method. FIG. 16 shows cross-section of a laser abrasive tip which functions as an accelerator of particles. This tip has a nozzle 1601, optical elements 1602 for delivery of the laser radiation (for example, an optical fiber) and a focusing element 1605 for focusing of the laser radiation on one or several abrasive particles 1603 and, optionally, on the liquid surrounding the particles. The shape of the nozzle may be optimized for better acceleration of the particles. For example, it can be a special nozzle shape to increase the speed of the particles. One or more abrasive particles 1603 is accelerated and directed toward a tissue 1604 by a mechanical pulse after the interaction with a laser pulse. In this case, the laser beam can be focused on a spot size close to the size of the particles in the range of 1-100 μm, preferably 10-30 μm. The particles can be delivered into the focus with a speed of about 1-100 cm/s and accelerated to a speed of 10-1000 m/s. A continuous flow of particles can be used for a CW laser. For a QCW laser, a discrete flow of particles can be used, and the laser pulse must be synchronized with placement of the particles into the focus of the laser beam. With the above-described tip, tissue removal occurs due to the mechanical interaction of the high-speed abrasive particles with the tissue, and not due to the laser ablation.

Increasing efficiency of the laser energy and power used in the above-described process can be achieved by partial processing of the treatment material. The optical system in the hand piece can be designed to form a non-uniform beam on the treated solid-state material or hard tissue. A view of the surface of the treated material or hard tissue is shown in FIG. 17. The treatment zone 1703, where all the material has to be removed, is split into two parts—1701 and 1702. The first part of the treatment zone 1701 is illuminated by the laser pulse with the fluence above the threshold of ablation. The second part of the treatment zone 1702 is not illuminated by the laser pulse or is illuminated with the fluence below or at about the threshold of ablation. This non-uniform ablation of the treatment tissue is achieved by a spatially non-uniform beam with fluence distribution similar to the pattern of ablation shown in FIG. 17, or by scanning a small beam to create an ablation pattern shown on FIGS. 8 and 17. The non-ablated material 1702 in the treatment zone 1703 can be removed using non-laser energy, such as mechanical, ultra sound or chemical energy. For example, it can be a rotary hand piece. It can also be solid or liquid particles possessing high kinetic energy, such as abrasive particles accelerated by a laser. For hard tissue ablation with a laser wavelength of 2700-3000 nm, the product of ablation is a flow of high-speed particles. Kinetic energy of these particles can be used to remove material in the second part of the treatment zone 1702. Chemical removal of the material in the second zone 1702 can be performed by acid etching (for example, by using phosphoric, hydrochloric or citric acids), or by using special compounds for removal of the decayed hard tissue (for example, Carisolv, Medi Team AB, Sweden). During laser ablation, the mechanical hardness and chemical resistance of material in the second zone 1702 is reduced due to micro-cracking and heating and can be easily removed if the size of the material between the ablated holes is not too large. Typically, this size must be in the range of 1-250 μm. The fill factor of first part 1701 relative to the entire treatment zone is in the range of 10-95%, preferably 30-85%, and most preferably 50-75%.

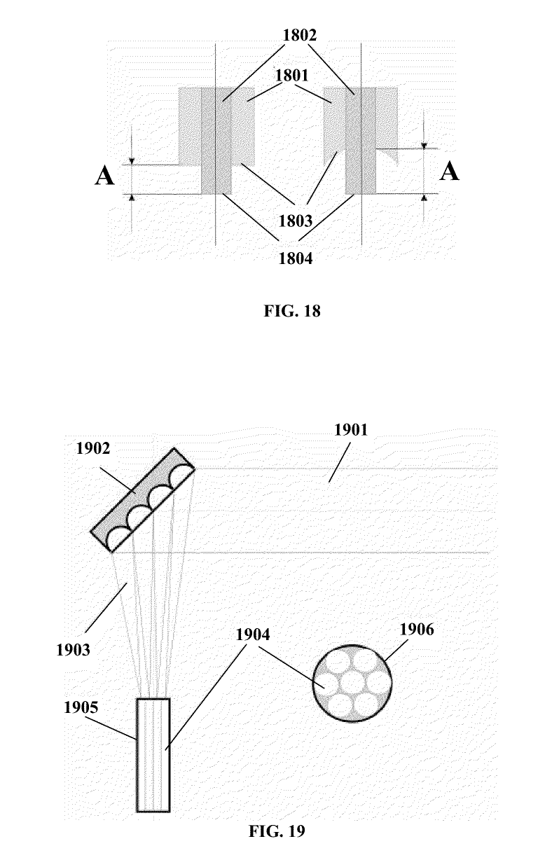

FIG. 18 shows one possible embodiment of a particle recycler and is not intended to be limiting in any way. The recycler is used for returning products of ablation into a treatment zone. The products of ablation may be the particles of the treated hard tissue. A recycler 1801 surrounds a fiber 1802 and may have a flat surface 1803 or a spherical surface 1804 facing the treatment zone. Upon reaching the particle recycler 1801, the products of ablation and shock waves are reflected back toward the treatment zone where they further destroy the tissue untreated with the direct laser beams. The distance A between the output end of the fiber and the surface of the particle recycler 1801 may be in the range of 0-1000 μm, preferably 50-200 μm. The volume of the space between the particle recycling surface and the surface of the treatment zone of the material must be maximal to provide for the leakage of the particles and pressure for the maximal concentration of the kinetic energy of the particles and of the acoustic energy of the shock wave on the portion of the treatment zone untreated with direct laser beams.

FIG. 19 shows a schematic drawing of the particle recycler and an optical system for creation of non-uniform light distribution on a treated hard tissue and is not intended to be limiting in any way. This optical system transforms input radiation into a plurality of focused beams onto a treatment zone. Laser radiation 1901 is reflected from a raster mirror 1902 comprised of an array of focusing minors and is separated into several beams 1903. Each beam 1903 is focused by each raster element into separate waveguides 1904 or directly on the treatment zone. A tip 1905 comprises of the waveguides 1904, which can be optical fibers. The space between the fibers 1904 is filled with a solid material which forms a particle recycler 1906. Laser radiation travels via the fibers 1904 and hits the treated hard tissue, creating products of ablation. Upon reaching the particle recycler 1906, the products of ablation are reflected back toward the treatment zone where they further destroy the hard tissue untreated with direct laser energy.

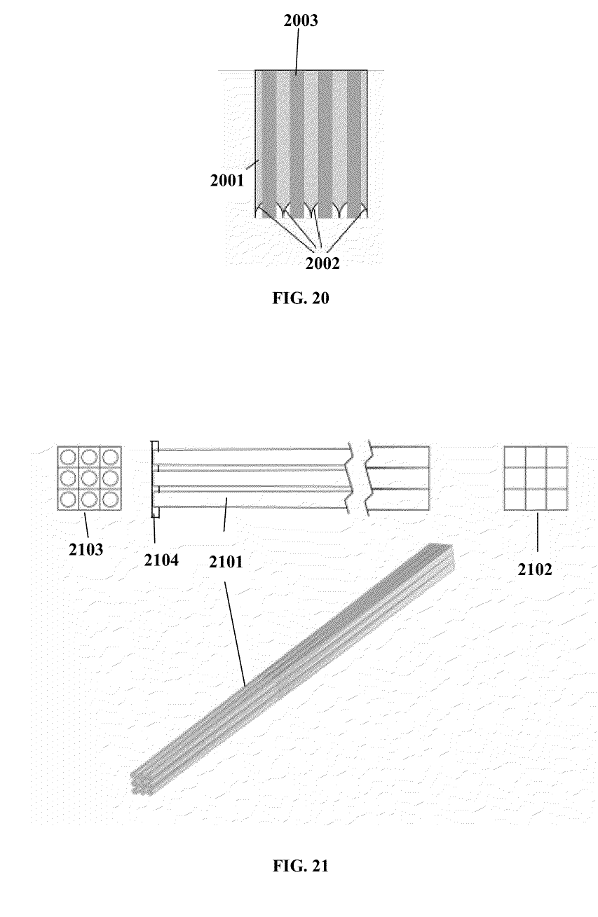

As shown in FIG. 20, a particle recycler 2001 may comprise of several spherical surfaces 2002 located around waveguides 2003. The spherical surfaces may have different radii for better particle and shock wave reflection and concentration.

FIG. 21 shows yet another embodiment of the tip. The tip comprises of at least two waveguides, forming a bundle of waveguides. The input aperture of the waveguides is confined with the laser beam to maximize coupling efficiency. The output end of the bundle has a space between the waveguides' ends filled by a particles reflector. The waveguides can be hollow waveguides, such as tapered holes in metal or ceramic. The output end can be covered by a thin plate which plays the role of a reflector of particles and shock waves from ablation. Such a plate can be made of diamond, sapphire or some other material transparent for laser radiation. It can be a disposable element of the tip. The waveguides can be fibers of a cylindrical or conical shape, as shown in FIG. 21.

While the invention has been described in conjunction with the detailed description thereof, the foregoing description is intended to illustrate and not to limit the scope of the invention, which is defined by the scope of the appended claims. Other aspects, advantages, and modifications are within the scope of the following claims. The use of “such as” and “for example” are only for the purposes of illustration and do not limit the nature or items within the classification.

What is claimed is:

1. A method for treating a material with optical radiation, the method comprising:
obtaining radiation from a radiation source with fluence and power density sufficient for ablating the material in a treatment zone having a first portion and a second portion;
applying the radiation to the treatment zone of the material to ablate the material in the first portion of the material in the treatment zone;
acoustically, mechanically or chemically removing the material from the second portion of the material in the treatment zone;
wherein the first portion is characterized by a fill factor relative to the treatment zone is ranging from 10% to 95%.

2. The method of claim 1, wherein the material is dental tissue or dental material.

3. The method of claim 1, further comprising forming an array of cavities in the first portion of the material in the treatment zone after the step of applying the radiation.

4. The method of claim 3, wherein the array is periodical.

5. The method as claimed in claim 1, wherein mechanically removing the material is accomplished by directing high speed particles onto the second portion of the material.

6. The method as claimed in claim 5, wherein the high speed particles are accelerated by the same radiation that ablates the first portion.

7. The method as claimed in claim 1, wherein applying the radiation to the treatment zone of the material to ablate the material results in formation of the high speed particles as products of ablation of the material in the first portion which is redirecting to second portion of treatment zone and mechanically destroying the second portion.
